(19) United States
(12) Patent Application Publication
(10) Pub. No.: US 2023/0329918 A1
(43) Pub. Date: Oct. 19, 2023

(54) PRESSURE-REDUCING DRESSING ADAPTED FOR SECURING MEDICAL EQUIPMENT

(71) Applicants: TAICEND TECHNOLOGY CO., LTD., Kaohsiung City (TW); CHI MEI MEDICAL CENTER, Tainan City (TW)

(72) Inventors: Fu-Chuan CHANG, Tainan City (TW); Ying-Li LEE, Tainan City (TW); Li-Fen CHUNG, Kaohsiung City (TW); Wen-Ling CHUANG, Tainan City (TW); Wen-Pei HUANG, Tainan City (TW); Hsiu-Hui WEI, Tainan City (TW); Gou-Don CHU, Tainan City (TW)

(21) Appl. No.: 18/076,894

(22) Filed: Dec. 7, 2022

(30) Foreign Application Priority Data
Apr. 19, 2022 (TW) 111114836

(51) Int. Cl. A61F 13/02

(52) U.S. Cl. CPC A61F 13/0243; A61F 13/0259; A61F 2013/00412

(57) ABSTRACT

A pressure-reducing dressing is adapted for securing medical equipment to the skin of a human body, and includes a skin contact pad adapted for locating the medical equipment, a water barrier laminate positioned on the skin contact pad, and a release sheet. The water barrier laminate includes a body layer and an adhesive layer formed on a side surface of the body layer that faces the skin contact pad and adhered to the skin. The body layer has a positioning portion and two divisional portions extending from the positioning portion and spaced apart from each other by a separating gap. The release sheet is attached to the adhesive layer of the water barrier laminate.

### CROSS-REFERENCE TO RELATED APPLICATIONS

This application claims priority of Taiwanese Patent Application No. 111114836, filed on Apr. 19, 2022.

### FIELD

The disclosure relates to a medical care product, more particularly to a pressure-reducing dressing adapted for securing medical equipment.

### BACKGROUND

Medical equipment such as needles, intravenous tubing sets and three-way stopcocks are used for intravenous injections, liquid deliveries, and other medical operations for a patient. In order to prevent a person from coming into contact with such medical equipment and injuring the patient, especially those who are confined to a wheelchair or are bedridden, the medical equipment typically needs to be partially attached to the skin of the patient. Currently, the practice of attaching the medical equipment to the skin of a patient with bandages and breathable tapes typically depends on the nursing staff's expertise, thereby resulting in low reliability.

Furthermore, when the medical equipment touches a patient's body for an extended period, pressure damage might occur and needs to be remedied.

### SUMMARY

Therefore, the object of the disclosure is to provide a pressure-reducing dressing adapted for securing medical equipment to the human body that can improve the reliability of fixation and reduce discomfort. According to the disclosure, a pressure-reducing dressing is adapted for securing medical equipment to the skin of a human body, and includes a skin contact pad adapted for locating the medical equipment, a water barrier laminate positioned on the skin contact pad, and a release sheet. The water barrier laminate includes a body layer and an adhesive layer formed on a side surface of the body layer that faces the skin contact pad and adhered to the skin. The body layer has a positioning portion and two divisional portions extending from the positioning portion and spaced apart from each other by a separating gap. The release sheet is attached to the adhesive layer of the water barrier laminate.

### BRIEF DESCRIPTION OF THE DRAWINGS

Other features and advantages of the disclosure will become apparent in the following detailed description of the embodiments with reference to the accompanying drawings, of which:

FIG. 1 is an exploded perspective view of a first embodiment of a pressure-reducing dressing of the disclosure adapted for securing medical equipment;

FIG. 2 is a schematic side view of the first embodiment, illustrating a skin contact pad, a water barrier laminate, a release sheet, and a backing layer of the first embodiment;

FIG. 3 is a schematic diagram illustrating the procedure for securing a medical three-way stopcock by the first embodiment;

FIG. 4 is a perspective view illustrating another implementation of the first embodiment;

FIG. 5 is an exploded perspective view of a second embodiment of a pressure-reducing dressing of the disclosure adapted for securing medical equipment; and

FIG. 6 is a schematic diagram illustrating the procedure for securing a needle by the second embodiment.

### DETAILED DESCRIPTION

Before the disclosure is described in greater detail, it should be noted that, where considered appropriate, reference numerals have been repeated among the figures to indicate corresponding or analogous elements.

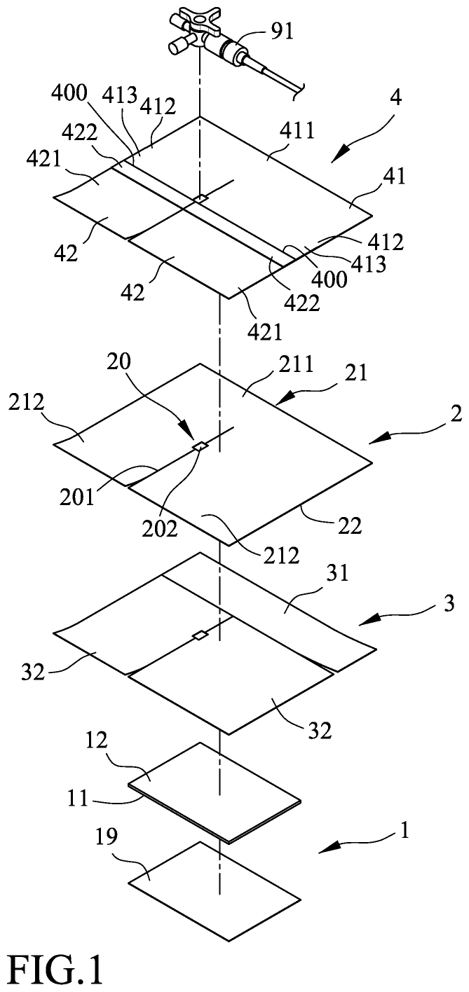

As shown in FIG. 1, the first embodiment of a pressure-reducing dressing according to the disclosure can be utilized in securing medical equipment on the skin of a human body, which includes a skin contact pad 1 made of foam material, a water barrier laminate 2 positioned on the skin contact pad 1, a release sheet 3 attached to the water barrier laminate 2, and a backing layer 4 attached to a side surface of the water barrier laminate 2 facing away from the skin contact pad 1. It should be noted that the medical three-way stopcock 91 is only used in the first embodiment as an example of medical equipment to illustrate how to secure the medical three-way stopcock 91 using the first embodiment.

Specifically, the skin contact pad 1 has a rectangular form and includes an interior contact surface 11 and an exterior surface 12. The inner contact surface 11 is configured for contact with the human body's skin, and the medical three-way stopcock 91 is positioned and fixed on the exterior surface 12. The inner contact surface 11 is attached with a release film 19 to preserve the surface characteristics of the interior contact surface 11. The skin contact pad 1 is made of low resilience foam material and has a thickness of 2˜15 mm, a density of 0.03˜0.45 g/cm³, an area of 10˜50 cm², a hardness (ASKER DUROMETER TYPE F) of 8˜50, and a rebound elasticity less than 10%. By selecting the proper material and parameters, skin contact pad 1 can be created to match the weight, form, and other specifications of the medical three-way stopcock 91. Besides preventing the medical three-way stopcock 91 from directly contacting the human body and causing discomfort, the skin contact pad 1 can reduce the pressure caused by the medical three-way stopcock 91 to the human body.

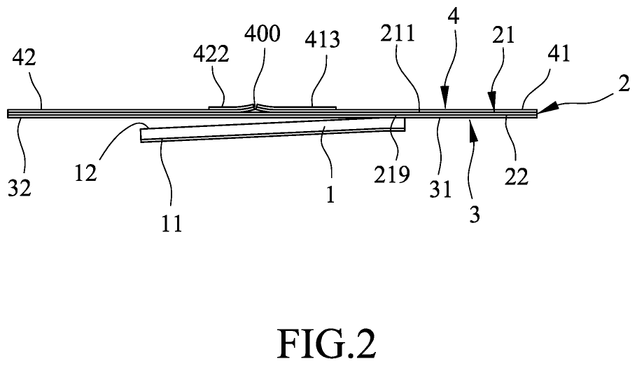

Referring to FIGS. 1 and 2, the water barrier laminate 2 is positioned on the exterior surface 12 of the skin contact pad 1 and includes a body layer 21 and an adhesive layer 22 formed on a side of the body layer 21 that faces the skin contact pad 1. In this embodiment, the body layer 21 is made of a transparent material, and includes a positioning portion 211 with a bonding zone 219 attached to a side edge of the skin contact pad 1 and two divisional portions 212 extending from the positioning portion 211 in a direction away from the bonding zone 219. The two divisional portions 212 are spaced apart to define a separating gap 20 therebetween. The separating gap 20 is configured as a slit, and has a straight slit body 201 extending linearly and an expanded slit part 202 laterally bulged on either side of the slit 201.

The release sheet 3 is attached to the adhesive layer 22 of the water barrier laminate 2, which includes a first section 31 corresponding in position to the positioning portion 211 and two second sections 32 corresponding in position to the divisional portions 212 respectively. The serial numbers first and second prefixed in the first section 31 and the second sections 32 also denote the order of peeling off during use.

The backing layer 4 has a first division zone 41 attached to the positioning portion 211 of the body layer 21 and portions of the divisional portions 212, and two second division zones 42 attached respectively to the two divisional portions 212 of the body layer 21 to form two separating slits 400. Each separating slit 400 is disposed between the first division zone 41 and a respective one of the second division zones 42. The first division zone 41 includes a first base part 411, two extension parts 412 each extending from the first base part 411 to lie over a part of a respective of the two divisional portions 212, and two first flaps 413 that are integrally connected to the two extension parts 412. Each second division zone 42 has a second base part 421 and two second flaps 422 that are integrally connected to the second base parts 421. Each second flap 422 cooperating with a respective one of the first flaps 413 to define a respective one of the separating slits 400 therebetween. The backing layer 4 is electrostatically attached to the body layer 21 of the water barrier laminate 2, thereby increasing the overall thickness and optimizing the operability of the dressing. Moreover, the separating slits 400 are spaced apart from the junction between the first section 31 and the second sections 32 of the release sheet 3, as depicted in FIG. 2, to make it simple to remove the backing layer 4 by the first flaps 413 and the second flaps 422, which are grabbed between fingers. That is, the separating slits 400 is farther away from the positioning portion 211 of the water barrier laminate 2 than the junction between the first section 31 and the second sections 32 of the release sheet 3.

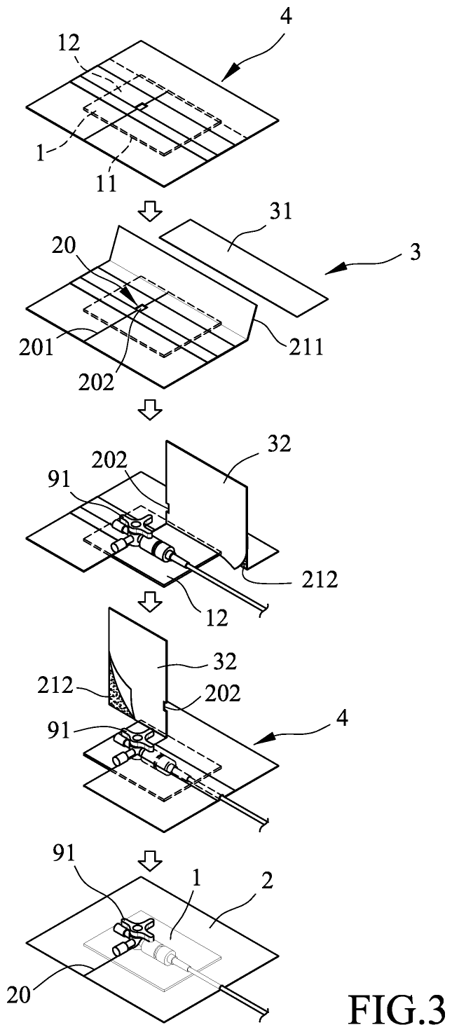

Referring to FIG. 3 and in conjunction with FIG. 1, when using the first embodiment to secure the medical three-way stopcock 91, the interior contact surface 11 of the skin contact pad 1 is first brought into contact with the skin of a human body at a place where the medical three-way stopcock 91 is to be positioned. Next, the first section 31 of the release sheet 3 is torn off so as to allow the positioning portion 211 of the body layer 21 of the water barrier laminate 2 to be adhered to the skin of the human body through the adhesive layer 22. At this time, both the second sections 32 of the release sheet 3 have not been peeled off so that the divisional portions 212 of the water barrier laminate 2 can be turned up along with the release sheet 3, and then the medical three-way stopcock 91 can be initially positioned on the exterior surface 12 of the skin contact pad 1. Subsequently, one of the second sections 32 of the release sheet 3 is peeled off, and then the respective divisional portion 212 is attached back onto a part of the medical three-way stopcock 91. The same operation applies to the other second section 32 and the corresponding divisional portion 212. Finally, the backing layer 4 is entirely torn off to complete securing the medical three-way Stopcock 91 on the skin of the human body with a part of the medical three-way stopcock 91 being exposed from the expanded slit part 202.

When completing the securing procedure, the medical three-way stopcock 91 is positioned between the water barrier laminate 2 and the skin contact pad 1. Besides, because the medical three-way stopcock 91 is partly inserted in the expanded slit part 202 so that it is confined between the divisional portions 212, a high-reliability fixation can be achieved through a simple operation. In addition, with the cushioning and pressure alleviating effect of the skin contact pad 1, the comfort of use can be significantly improved.

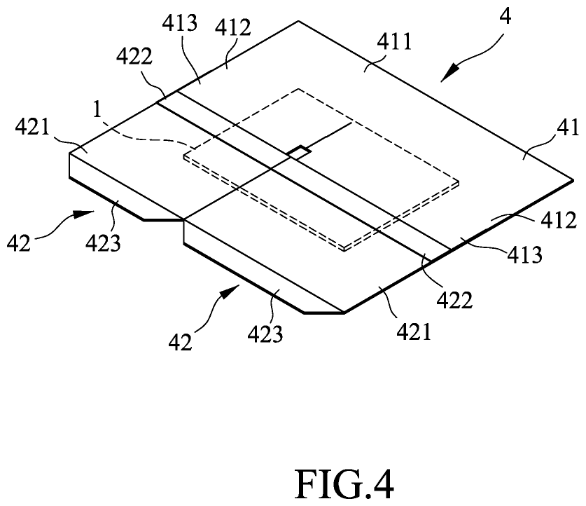

Referring to FIG. 4, another implementation of the first embodiment is illustrated, the main difference is that each of the second division zones 42 of the backing layer 4 further has a tear tab 423 opposite to the second flaps 422 and extending from the corresponding second base 421 in a direction away from the corresponding second flap 422 for easy removal of the respective second division zone 42.

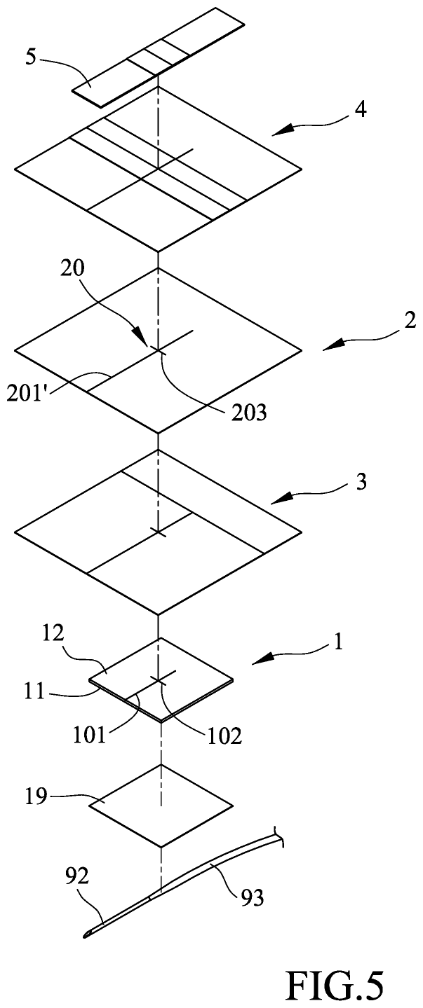

As shown in FIG. 5, the second embodiment has a structure similar to that of the first embodiment. The main difference between this embodiment and the previous embodiment resides in that: the skin contact pad 1 has an elongated aperture positioned corresponding in position to the separating gap 20. The elongated aperture has a long slit part 101 and a short slit part 102 that intersects perpendicularly with the long slit part 101 and that is shorter than the long slit part 101. In addition, the separating gap 20 is configured as a slit, and has a long slit part 201′ extending linearly and a transverse slit 203 intersecting perpendicularly with the long slit part 201′ and positioned in registration with the short slit part 102. The second embodiment further includes an auxiliary sealing piece 5 that is attached to the water barrier laminate 2 and that seals the separating gap 20 and is functionally comparable to the water barrier laminate 2.

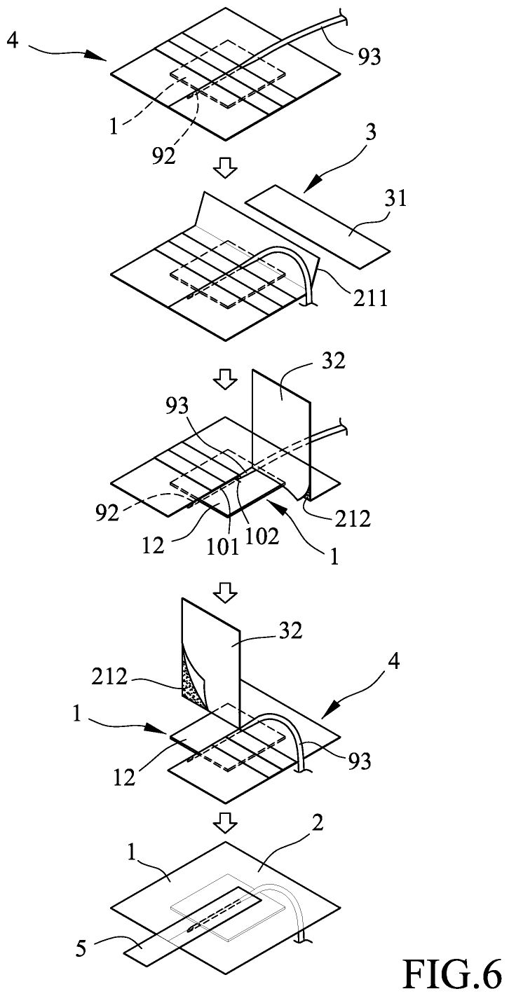

Referring to FIG. 6 and in conjunction with FIG. 5, in the second embodiment, the medical equipment is a needle 92. To meet the needle-securing needs, the skin contact pad 1 in this embodiment is made of polyurethane high-absorbent foam material. The polyurethane high-absorbent foam has a thickness of 0.5-5.0 mm, a density of 0.15-0.45 g/cm³, an area of 10-50 cm², and a water absorption ratio of 7-15 times. In addition to primarily reducing pressure and improving comfort, by choosing the material and the parameters of the skin contact pad 1, liquids including tissue fluid, injection fluid and blood can be absorbed thereby.

When utilizing the second embodiment to fasten the needle 92 on the skin of a human body, by allowing the needle 92 to extend along the aperture 101, the skin contact pad 1 can be positioned to sufficiently cover the needle 92. At this time, in case the needle 92 is connected to tubing set 93 of an intravenous fluid bottle, the tubing set 93 can be arranged to extend outwardly through the transverse aperture 102. Subsequently, the steps including the attachment of the water barrier laminate 2, the attachment of the divisional portions 212, and the removal of the backing layer 4 are the same as those in the first embodiment. After removing the backing layer 4, the auxiliary sealing piece 5 will be attached to the water barrier laminate 2, totally closing the separating gap 20, thus reliably securing the needle 92.

The second embodiment achieves the same effect as the first embodiment. Since the skin contact pad 1 is made of high-absorbent material, and since using the auxiliary sealing piece 5 and the water barrier laminate 2 prevents extravasations, in addition to alleviating contact pressure and enhancing comfort, tissue fluid, injection fluid, or even blood seep out of the needle 92 can be absorbed by the skin contact pad 1.

To sum up, the embodiments of the pressure-reducing dressing of the disclosure for securing medical equipment can apply to various forms of medical equipment, achieving a better securing effect and anti-seepage effect. Therefore, the object of the disclosure can be achieved indeed.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A pressure-reducing dressing adapted for securing medical equipment to the skin of a human body, comprising:
a skin contact pad including an interior contact surface adapted to be placed on said skin of a human body, and an exterior surface opposite to said interior contact surface and adapted for locating the medical equipment; and
a water barrier laminate positioned on said exterior surface of said skin contact pad and including
a body layer having a positioning portion with a bonding zone attached to at least a part of said skin contact pad, and two divisional portions extending from said positioning portion in a direction away from said bonding zone and spaced apart from each other by a separating gap, and
an adhesive layer formed on a side surface of said body layer that faces said skin contact pad, said adhesive layer being adapted to be adhered to said skin of human body so as to confine the medical equipment between said skin contact pad and said water barrier laminate; and
a release sheet attached to said adhesive layer of said water barrier laminate and having a first section corresponding in position to said positioning portion and two second sections corresponding in position to said two divisional portions, respectively.

2. The pressure-reducing dressing as claimed in claim 1, wherein said separating gap of said body layer of said water barrier laminate is configured as a slit and has a straight slit body extending linearly and an expanded slit part laterally bulged on either side of said slit body.

3. The pressure-reducing dressing as claimed in claim 1, wherein said skin contact pad is made of low-resilience foam material and has a thickness of 2˜15 mm, a density of 0.03˜0.45 g/cm³, an area of 10˜50 cm², a hardness (ASKER DUROMETER TYPE F) of 8˜50, and a rebound elasticity less than 10%.

4. The pressure-reducing dressing as claimed in claim 1, wherein said skin contact pad has an elongated aperture configured as a slit and corresponding in to said separating gap.

5. The pressure-reducing dressing as claimed in claim 4, wherein said elongated aperture has a long slit part and a short slit part that intersects perpendicularly with said long slit part and that is shorter than said long slit part, said separating gap of said body layer being configured as a slit and having a longitudinal slit part extending linearly and a transverse slit part intersecting perpendicularly said longitudinal slit part and located in registration with said short slit part.

6. The pressure-reducing dressing as claimed in any one of claim 1, wherein said skin contact pad is made of polyurethane high-absorbent foam material and has a thickness of 0.5˜5.0 mm, a density of 0.15˜0.45 g/cm³, an area of 10˜50 cm², and an absorption rate of 7˜15 times.

7. The pressure-reducing dressing as claimed in any one of claim 1, further comprising a backing layer attached to a Side surface of said water barrier laminate facing away from said skin contact pad.

8. The pressure-reducing dressing as claimed in claim 7, wherein:
said backing layer includes
a first division zone attached to said positioning portion of said body layer and portions of said two divisional portions, and
two second division zones attached respectively to said two divisional portions of said body layer to form two separating slits each disposed between said first division zone and a respective one of said second division zones;
said first division zone has a first base part, two extension parts each extending from said first base part to lie over a part of a respective one of said divisional portions, and two first flaps that are integrally attached to said two extension parts; and
each of said second division zones has a second base part and two second flaps that are integrally attached to said second base parts, each of said second flaps cooperating with a respective one of said first flaps to define a respective one of said separating slits therebetween.

9. The pressure-reducing dressing as claimed in claim 8, wherein said separating slits are spaced apart from a junction between said first section and said second sections of said release sheet.

10. The pressure-reducing dressing as claimed in claim 8, wherein each of said second division zones of said backing layer further has a tear tab opposite to said second flaps and extending from a corresponding one of said second base parts in a direction away from a corresponding one of said second flaps.
